(19) United States
(12) Patent Application Publication
(10) Pub. No.: US 2007/0143880 A1
(43) Pub. Date: Jun. 21, 2007

(54) Methods for Introducing Into a Plant a Polynucleotide of Interest

(75) Inventors: Dennis L. Bidney, Urbandale, IA (US); Sheila E. Maddock, Johnston, IA (US); Xinli E. Wu, Johnston, IA (US); Zuo-Yu Zhao, Johnston, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 11/427,371

(22) Filed: Jun. 29, 2006

Related U.S. Application Data
(60) Provisional application No. 60/751,385, filed on Dec. 16, 2005.

(51) Int. Cl. A01H 1/00; C12N 15/82

(52) U.S. Cl. 800/294

(57) ABSTRACT

The present invention provides methods and compositions which deliver Agrobacterium via microinjection directly into the embryo sac. At the time of injection, the embryo sac can comprise an egg cell, or alternatively, the embryo sac can be fertilized and comprise either a zygote or an embryo. Once inside the embryo sac, the Agrobacterium harboring a T-DNA having a polynucleotide of interest can express of the polynucleotide of interest in the plant. Further, the Agrobacterium can transfer the T-DNA having the polynucleotide of interest to the plant nucleus to produce a transformed plant. The polynucleotide of interest may be stably integrated into the genome of the egg cell, zygote, embryo, or endosperm, and any tissue, plant part, and/or plant generated therefrom.

### CROSS-REFERENCE TO RELATED APPLICATIONS

This application claims the benefit of U.S. Application Ser. No. 60/751,385 filed Dec. 16, 2005, which is herein incorporated by reference in its entirety.

### FIELD OF THE INVENTION

The invention relates to the field of molecular biology, more particularly to the field of plant transformation.

### BACKGROUND

Many plant transformation systems involve extensive callus-based culture and selection. These methodologies are time consuming and may increase the likelihood that somaclonal variants will arise that exhibit undesirable agronomic characteristics. The use of developmentally organized explants as targets for transformation minimizes tissue culture steps but increases the likelihood that chimeric plants are produced. Targeting gametes, zygotes or early stage embryos in embryo sacs for transformation is a potential solution to these problems. Although the production of gametes and zygotes by plants is well-understood, reproducible methods for in vitro manipulation and transformation of these cells are needed to provide improved methods of transforming plants.

### SUMMARY

Methods and compositions are provided which deliver Agrobacterium via microinjection directly into the embryo sac. At the time of injection, the embryo sac can comprise an egg cell, or alternatively, the embryo sac can be fertilized and comprise either a zygote or an embryo. Once inside the embryo sac, the Agrobacterium harboring a T-DNA having a polynucleotide of interest can express the polynucleotide of interest in the plant. Further, the Agrobacterium can transfer the T-DNA having the polynucleotide of interest to the plant nucleus to produce a transformed plant. The polynucleotide of interest may be stably integrated into the genome of the egg cell, zygote, embryo, or endosperm, and any tissue, plant part, and/or plant generated therefrom.

### BRIEF DESCRIPTION OF THE DRAWINGS

FIG. 1A (Left) shows an immature kernel section (slab) from a 1-day post-pollinated immature kernel containing an embryo sac that was microinjected with Agrobacterium. The T-DNA within the injected Agrobacterium comprises Actin promoter::GUS (PHP22462). Following injection of the Agrobacterium, expression of GUS gene in the embryo sac (stained structure at the bottom of the section, arrow) indicates that the functional Agrobacterium has been delivered and the GUS gene on the T-DNA was expressed. Bar=0.5 mm.

FIG. 1 B (Right) shows an embryo/endosperm structure derived from a 1-day post-pollinated microinjected nucellar slab after 21 days in vitro culture. The nucellar slab, isolated from a transgenic plant stably transformed with PHP10006 (Ubi::FRT1::GFP::35S::MOPAT::FRT1::GUS::FRT5), was injected with Agrobacterium containing PHP17797 (RB::Ubi-FRT1::FLPm::35S::Bar::FRT5::LB). The FLP enzyme expressed from PHP17797 mediated excision of the DNA between the FRT1 sites in PHP10006 in the injected slab, thereby creating an operable linkage of the promoterless GUS gene to the Ubi promoter, resulting in GUS expression. The blue spot (arrow) shows the GUS expression in the embryo area, demonstrating T-DNA transfer and gene expression from the injected Agrobacterium. Bar=1 mm.

### DETAILED DESCRIPTION

Methods and compositions which deliver Agrobacterium via microinjection directly into the embryo sac include, but are not limited to the following:
1. A method for expressing a polynucleotide of interest in a plant comprising
   a) providing an embryo sac from the plant;
   b) injecting into said embryo sac a composition comprising an effective concentration of an Agrobacterium comprising a T-DNA comprising the polynucleotide of interest operably linked to a promoter active in the plant, wherein said Agrobacterium is capable of T-DNA transfer into a plant cell.
2. The method of 1, further comprising recovering from said embryo sac a transgenic plant having said polynucleotide of interest stably integrated into its genome.
3. The method of any one of 1 or 2, wherein said embryo sac comprises a fertilized embryo sac.
4. The method of 3, wherein said fertilized embryo sac comprises an embryo.
5. The method of 3, wherein said fertilized embryo sac comprises a zygote.
6. The method of 3, wherein said fertilized embryo sac comprises a one-day post-pollination embryo sac.
7. The method of 1, wherein said embryo sac comprises an egg cell and said method further comprises
   a) fertilizing said egg cell from step (b) with a plant sperm cell; and,
   b) recovering a plant having said polynucleotide of interest stably integrated into its genome.
8. The method of 1, wherein said plant comprises a monocot.
9. The method of 8, wherein said monocot is maize, barley, millet, wheat, or rice.
10. The method of 1, wherein said plant comprises a dicot.
11. The method of 10, wherein said dicot is soybean, canola, alfalfa, sunflower, safflower, tobacco, Arabidopsis, or cotton.
12. The method of 1, wherein said Agrobacterium comprises Agrobacterium tumefaciens.
13. A method of introducing an Agrobacterium into a plant, said method comprising
   a) providing an embryo sac from the plant;
   b) injecting into said embryo sac a composition comprising an effective concentration of an Agrobacterium, wherein said Agrobacterium is capable of T-DNA transfer into a plant cell.
14. The method of 13, wherein said Agrobacterium comprises a T-DNA comprising a polynucleotide of interest operably linked to a promoter active in the plant.
15. The method of 14, further comprising recovering from said embryo sac a transgenic plant having said polynucleotide of interest stably integrated into its genome.
16. The method any one of 13-15, wherein said embryo sac comprises a fertilized embryo sac.
17. The method of 16, wherein said fertilized embryo sac comprises an embryo.
18. The method of 16, wherein said fertilized embryo sac comprises a zygote.
19. The method of 16, wherein said fertilized embryo sac comprises a one-day post-pollination embryo sac.
20. The method of 14, wherein said embryo sac comprises an egg cell and said method further comprises
   a) fertilizing said egg cell of step (b) with a plant sperm cell; and,
   b) recovering the plant having said polynucleotide of interest stably integrated into its genome.
21. The method of any one of 13-15, or 20, wherein said plant comprises a monocot.
22. The method of 21, wherein said monocot is maize, barley, millet, wheat, or rice.
23. The method of any one of 13-15, or 20, wherein said plant comprises a dicot.
24. The method of 23, wherein said dicot is soybean, canola, alfalfa, sunflower, safflower, tobacco, Arabidopsis, or cotton.
25. The method of any one of 13-15, or 20, wherein said Agrobacterium comprises Agrobacterium tumefaciens.
26. A method to identify a fertilized plant embryo sac comprising
   a) providing pollen from a first plant comprising a polynucleotide encoding a visual marker operably linked to a promoter active in said pollen or in an embryo sac;
   b) providing a population of unfertilized seed, each seed comprising an embryo sac;
   c) contacting said seed with said pollen; and
   d) identifying the fertilized embryo sac expressing said visual marker.
27. The method of 26, wherein the visual marker is expressed in the central cell of the embryo sac, the zygote of the embryo sac, the pollen, or the pollen tube.
28. The method of any one of 26-27, wherein said visual marker is a fluorescent protein.
29. The method of 28, wherein the fluorescent protein is selected from the group consisting of a yellow fluorescent protein (YFP), a green fluorescent protein (GFP), a cyan fluorescent protein (CFP), and a red fluorescent protein (RFP).
30. The method of 29, wherein said visual marker is encoded by a polynucleotide having maize preferred codons.
31. The method of 30, wherein said visual marker comprises GFPm, AmCyan, DsYellow, or ZsRed.
32. The method of any one of 26-31, wherein said fertilized embryo sac is identified at about one day post-pollination or prior to one day post-pollination.
33. The method of any one of 26-31, wherein said fertilized plant embryo is from a monocot.
34. The method of 33, wherein said monocot is maize, barley, millet, wheat, or rice.
35. The method of any one of 26-31, wherein said fertilized plant embryo is from a dicot.
36. The method of 35, wherein said dicot is soybean, canola, alfalfa, sunflower, safflower, tobacco, Arabidopsis, or cotton.

Microinjection of DNA into embryos sacs and zygotes of plants has been investigated for a number of years in corn and other crops. However, no method has provided stably transformed plants at a high frequency. The difficulties could result from the digestion of bare foreign DNA by plant endonuclease enzymes, or arise from the difficulty in targeting the microinjection into the nucleus of the single-cell zygote. Methods and compositions are provided which deliver Agrobacterium via microinjection directly to the embryo sac. At the time of injection, the embryo sac can comprise an egg cell, or alternatively, the embryo sac can be fertilized and comprise either a zygote or an embryo. Once inside the embryo sac, the Agrobacterium harboring a T-DNA having a polynucleotide of interest can transfer the polynucleotide of interest to the plant nucleus allowing expression and/or integration of the polynucleotide of interest in the plant. In specific examples, the polynucleotide of interest is stably integrated into the genome of the egg cell, zygote, embryo, or endosperm, and any tissue, plant part, and/or plant generated therefrom.

Novel methods and compositions for introducing Agrobacterium into a plant are provided. In particular, the novel methods comprise injecting into a plant embryo sac a composition comprising an effective concentration of a biologically active Agrobacterium which is capable of T-DNA transfer into a cell. The methods thereby provide an efficient means for delivering a polynucleotide of interest into an unfertilized or a fertilized embryo sac, and further provide an effective method for the expression and/or integration of a polynucleotide of interest in a plant.

An embryo sac is typically an eight-nucleate female gametophyte. The embryo sac arises from the megaspore by successive mitotic divisions and comprises three antipodal cells, the egg cell, two synergids, and the central cell, which contains the two polar nuclei. The polar nuclei unite with the nucleus of a sperm cell in a triple fusion. In certain seeds, including, cereal seeds, the product of this triple fusion develops into the 3n endosperm.

An ovule is the structure in seed plants which contains the female gametophyte. The ovule is comprised of the nucellus which is surrounded by one or two integuments, and it is attached to the placenta by a stalk known as the funiculus. A nucellus is the tissue within the ovule in which the female gametophyte (i.e., the embryo sac) develops. The nucellus is the maternal tissue that is adjacent to the embryo sac.

The embryo sac employed in the methods can be unfertilized. In other methods, a fertilized embryo sac is injected with the Agrobacterium. A fertilized embryo sac is an embryo sac following the fusion of a sperm cell with the egg cell and/or the fusion of a sperm cell with the central cell. Typically, a fertilized embryo sac results from a double fertilization, wherein a first sperm cell fuses with the egg cell and a second sperm cell fuses with the central cell. The injection of the Agrobacterium into the fertilized egg sac can occur at any time following fertilization including at about less than 1, 1, 2, 3, 4, 5, 6, 7, 8, 9, 10, 11, 12, 13, 14, 15 or more days post-pollination.

An egg cell is a female gamete cell. A zygote is a fertilized embryo at the one-cell stage of development beginning upon fertilization of the egg cell with the sperm and concludes upon cleavage of the zygote into a two-cell embryo.

An embryo comprises any early stage embryo. An early stage embryo encompasses all embryonic stages that begin upon fertilization of the egg cell to produce the zygote and extends through the 2-cell stage, the 4-cell stage, and the 8 cell stage.

The embryo sacs employed in the methods can be obtained using a variety of methods. An isolated embryo sac is separated from a portion of the plant but continues to retain the structural integrity of the embryo sac. In specific examples, the isolated embryo sac is surrounded by the ovary cell wall and/or the nucellus. In one method, the plant embryo sac is isolated via micromanipulation, for example, the embryo sac can be isolated by serially sectioning the ovaries. The thickness of the sections varies from species to species depending on the size of the ovule, sections of less than about 150 μm, 150 μm, 160 μm, 170 μm, 180 μm, 190 μm, 200 μm, 210 μm, 220 μm, 230 μm, 240 μm, 250 μm, 275 μm, 300 μm, 325 μm, 350 μm, 375 μm, 400 μm, 450 μm, 500 μm, and up to the thickness of the ovule can be used. For example, the thickness of sections for maize typically ranges between about 200 μm to about 300 μm. The isolated ovary sections prepared by this method comprise embryo sacs surrounded by tissue from nucellus and the ovary wall. The intact embryo sacs are contained between thin layers of nucellus and are visible using a stereomicroscope. The sections containing the embryo sacs can be readily manipulated with forceps. Additional details regarding the method of isolation can be found, for example, in U.S. Pat. No. 6,300,543 and Laurie et al. (1999) In Vitro Cell. Dev. Biol. Plant 35:320-325. Additional methods for isolation of embryo sacs include dissection, micromanipulation, enzymatic maceration or any combination of methods. See, for example, Wagner et al. (1989) Plant Sci 59:127-132; Kranz et al. (1993) Plant Cell 5:739; Allington et al. (1985) The Experimental Manipulation of the Ovule Tissue, Longman, N.Y., 39-51; and, Theunis et al. (1991) Sex Plant Reprod 4:145-154.

Various methods for identifying fertilized plant embryo sacs can be employed. In one example, the identification of a fertilized plant embryo sac comprises contacting pollen from a plant comprising a polynucleotide encoding a visual marker operably linked to a promoter that is active in pollen or in an embryo sac with a population of unfertilized seed, each seed comprising an embryo sac. Fertilized embryos expressing the visual marker can then be identified.

Pollen refers to the male gametophyte. Any means of contacting the pollen to the unfertilized seed can be used. For example, the pollen can be applied either artificially or naturally to the ovule (stigma) of a plant of interest.

Any promoter active in the pollen or the embryo sac can be used in this method. Such promoters include, but are not limited to, constitutive promoters, promoters that are active in the central cell of the embryo sac, promoter that are active in the zygote of the embryo sac, promoters that are active in the pollen, and/or promoters that are active in the pollen tube. A female-preferential promoter refers to a promoter having transcriptional activity only, or primarily, in one or more of the cells or tissues of a female reproductive structure of a plant. Promoters active in these tissues are known. See, for example, U.S. Pat. Nos. 6,576,815, 6,452,069, Potenza et al. (2004) In Vitro Cell Dev Biol 40:1-22, and Hamilton et al. (1998) Plant Mol Bio 38:663-9.

While any visual maker can be used to identify the fertilized embryo, in one example, the visual marker is a fluorescent protein. Such fluorescent proteins include but are not limited to yellow fluorescent protein (YFP), green fluorescent protein (GFP), cyan fluorescent protein (CFP), and red fluorescent protein (RFP). In still other examples, the visual marker is encoded by a polynucleotide having maize preferred codons. In further examples, the visual marker comprises GFPm, AmCyan, DsYellow, or ZsRed. See, Wenck et al. (2003) Plant Cell Rep. 22:244-251.

In one non-limiting example, the embryos can be isolated from maize ears pollinated by a transgenic line comprising a fluorescent marker expressed in pollen. The putative pollinated embryos can be identified by screening for the pollen marker. The method can be used to identify fertilized embryo sac at any time following fertilization including at about less than 1, 1, 2, 3, 4, 5, 6, 7, 8, 9, 10, 11, 12, 13, 14, 15 or more days post-pollination.

The embryo sac used can be from any plant species, including, but not limited to, monocots and dicots. Examples of plant species of interest include, but are not limited to, corn (Zea mays), Brassica sp. (e.g., B. napus, B. rapa, B. juncea), castor, palm, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), Arabidopsis thaliana, oats (Avena spp.), barley (Hordeum spp.), leguminous plants such as guar beans, locust bean, fenugreek, garden beans, cowpea, mungbean, fava bean, lentils, and chickpea, vegetables, ornamentals, grasses, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and Cucumis species such as cucumber (C. sativus), cantalope (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii), Western hemlock (Tsuga canadensis), Sitka spruce (Picea glauca), redwood (Sequoia sempervirens), true firs such as silver fir (Abies amabilis) and balsam fir (Abies balsamea), and cedars such as Western red cedar (Thuja plicata) and Alaska yellow-cedar (Chamaecyparis nootkatensis).

The embryo sac employed in the methods can be unfertilized. In this method, the injected embryo sac comprises an egg cell. Before, during, and/or following injection of the embryo sac, the egg cell is fertilized with sperm cell from the plant (see, for example, Krantz “In Vitro Fertilization, Ch. 7, pp. 143-166 in Current Trends in the Embryology of Angiosperms, Bhojwani and Soh (Eds.) 2001 Kluwer Academic Publishers; and U.S. Pat. No. 6,300,543). Plants can be recovered from the in vitro fertilized and injected embryo sac.

In still other examples, the developing central cell and/or endosperm in the embryo sac is targeted for transformation by microinjection. Such methods allow one to evaluate the strength of regulatory sequences, such as a specific promoter, in this tissue, and/or to evaluate genes, for example genes involved in development or specific tissues such as genes involved in early seed processes, embryo or derived tissues and/or endosperm or derived tissues. In this method, embryo sacs are isolated, before pollination or from a fertilized plant, and microinjected with the Agrobacterium having the polynucleotide of interest. The derived endosperm cells are allowed to develop in vitro, while monitoring for gene delivery, expression, and/or phenotypic effects. The foreign DNA, for example, may be a reporter gene such as GUS operably linked to a promoter to be tested. GUS expression is assayed or quantified by well known methods such as Northern blot, PCR or Western blot techniques. See, for example, Ausubel et al. eds. (1990) Current Protocols in Molecular Biology, Wiley Interscience. Based on the level of expression of the polynucleotide of interest in the endosperm, a prediction can be made as to the whether the selected regulatory sequence will drive sufficient expression of the foreign gene to modify the phenotype of the seed.

A variety of bacterial strains may be used to introduce one or more polynucleotides of interest into a plant. In one method the Agrobacterium employed harbors at least one polynucleotide of interest operably linked to a promoter active in a plant which is located between the T-DNA borders, wherein the Agrobacterium employed is capable of T-DNA transfer into a plant cell. The Agrobacterium employed in the methods contains the necessary genetic elements for T-DNA transfer into a plant cell. A number of wild-type and disarmed strains of Agrobacterium tumefaciens and Agrobacterium rhizogenes harboring T-DNA, Ti or Ri plasmids can be used. The Agrobacterium hosts typically comprise disarmed Ti and Ri plasmids that do not contain the oncogenes which cause tumorigenesis or rhizogenesis, respectfully.

Any strain of Agrobacterium can be used, so long as it is biologically active and harbors a T-DNA comprising the polynucleotide of interest. A number of references review Agrobacterium-mediated transformation in monocots and dicots. See, for example, Hellens et al. (2000) Trends Plant Sci 5:446-451; Hooykaas (1989) Plant Mol. Biol. 13:327-336; Smith et al. (1995) Crop Sci 35:301-309; Chilton (1993) Proc. Natl. Acad. Sci. (USA) 90:3119-3210; and Moloney et al. (1993) In: Monograph Theor. Appl. Genet., N.Y., Springer Verlag 19:148-167.

Agrobacterium strains of interest can be wild type or derivatives thereof which have alterations that increase transformation efficiency. Strains of interest include, but are not limited to, A. tumefaciens strain C58, a nopaline-type strain (Deblaere et al. (1985) Nucleic Acids Res. 13:4777-4788); octopine-type strains such as LBA4404 (Hoekema et al. (1983) Nature 303:179-180); or succinamopine-type strains e.g., EHAL01 or EHAL05 (Hood et al. (1986) J. Bacteriol. 168:1291-1301); A. tumefaciens strain A281 (U.S. Patent Publication No. 20020178463); GV2260 (McBride et al. (1990) Plant Mol. Biol. 14:269-276); GV3100 and GV3101 (Holsters et al. (1980) Plasmid 3:212-230); A136 (Watson et al. (1975) J. Bacteriol. 123:255-264); GV3850 (Zambryski et al. (1983) EMBO J. 2:2143-2150); GV3101::Pmp90 (Koncz et al. (1986) Mol. Gen. Genet 204:383-396); and, AGL-1 (Lazo et al. (1991) Biotechnology 9:963-967). The methods and use of these strains for plant transformation has been reported.

Transfer DNA or T-DNA comprises a genetic element that is capable of integrating a polynucleotide contained within its borders into another polynucleotide. The T-DNA can comprise the entire T-DNA, but need only comprise the minimal sequence necessary for cis transfer, typically the right or left border is sufficient. The T-DNA can be synthetically derived or can be from an A. rhizogene Ri plasmid or from an A. tumefaciens Ti plasmid, or functional derivatives thereof. Any polynucleotide to be transferred, for example a recombinase, a polynucleotide of interest, a recombination site, a restriction site, a recognition site, a sequence tag, a target site, a transfer cassette and/or a marker sequence may be positioned between the left border sequence and the right border sequence of the T-DNA. The sequences of the left and right border sequences may or may not be identical and may or may not be inverted repeats of one another. It is also possible to use only one border, or more than two borders, to accomplish transfer of a desired polynucleotide.

Various plasmids are available comprising T-DNAs that can be employed in the methods. For example, many Agrobacterium employed for the transformation of dicotyledonous plant cells contain a vector having a DNA region originating from the virulence (vir) region of the Ti plasmid. The Ti plasmid originated from A. tumefaciens, and the polynucleotide of interest can be inserted into this vector. Alternatively, the polynucleotide of interest can be contained in a separate plasmid which is then inserted into the Ti plasmid in vivo, in Agrobacterium, by homologous recombination or other equivalently resulting processes. See, for example, Herrera-Esterella et al. (1983) EMBO J. 2:987-995 and Horch et al. (1984) Science 223:496-498.

A vector has also been developed which contains a DNA region originating from the virulence (vir) region of Ti plasmid pTiBo542 (Jin et al. (1987) J. Bacteriol. 169:4417-4425) contained in a super-virulent A. tumefaciens strain A281 showing extremely high transformation efficiency. This particular vector includes regions that permit vector replication in both E. Coli and Agrobacterium. See, Hood et al. (1984) Bio/Tech 2:702-709; Komari et al. (1986) Bacteriol 166:88-94. This type of vector is known superbinary vector (see European Patent Application 0604662A1). Examples of superbinary vectors include pTOK162 and pTIBo542 (U.S. Patent Publication No. 2002178463 and Japanese Laid-Open Patent Application no. 4-222527); pTOK23 (Komari et al. (1990) Plant Cell Rep. 9:303-306); pPHP10525 (U.S. Pat. No. 6,822,144), see, also Ishida et al. (1996) Nat Biotech. 14:745-750.

Additional transformation vectors comprising T-DNAs that can be used further include, but are not limited to, pBIN19 (Bevan et al. (1984) Nucleic Acids Res 12:8711-8721); pC22 (Simoens et al. (1986) Nucleic Acids Res 14:8073-8090); pGA482 (An et al. (1985) EMBO J. 4:277-284); pPCV001 (Koncz et al. (1986) Mol. Gen. Genet. 204:383-396); PCGN1547 (McBride et al. (1990) Plant Mol. Biol. 14:269-276); pJJ1881 (Jones et al. (1992) Transgenic Res. 1:285-297); pPzPl 11 (Hajukiewicz et al. (1994) Plant Mol. Biol. 25:989-994); and, pGreenOO29 (Hellens et al. (2000) Plant Mol. Biol. 42:819-832).

The polynucleotide may be inserted into a restriction site in the T-DNA region of the transformation vector, and the desired recombinant vector may be selected by using an appropriate selection marker, such as drug resistance and the like contained on the plasmid employed. General molecular biological techniques used are provided, for example, by Sambrook et al. (eds.) (1989) Molecular Cloning: A Laboratory Manual, Cold Spring Harbor Laboratory Press, Cold Spring Harbor, N.Y.

The introduction of a vector into a bacterium belonging to genus Agrobacterium such as Agrobacterium tumefaciens can be carried out by various conventional methods including, the triple cross method (Ditta (1980) Proc. Natl. Acad. Sci. USA 77:7347-7351) and the methods disclosed in Ishida et al. (1996) Nat Biotech 14:745-750 and in U.S. Patent Publication No. 20010054186. The Agrobacterium having the desired transformation plasmid can be isolated based on the use of a selectable marker incorporated into the vector.

Methods to determine if an Agrobacterium is capable of T-DNA transfer into a plant cell are known. For example, Agrobacterium harboring the vector carrying the T-DNA comprising an appropriate marker is contacted to the appropriate plant tissue. Agrobacterium capable of T-DNA transfer can be identified by their ability to transfer the T-DNA, and the accompanying marker, into the plant tissue. Methods to assay for various markers are routine. See, for example, Shurvinton et al. (1992) Proc. Natl. Acad. Sci. 89:11837-11841; Mysore et al. (1998) Mol. Plant Microb Interact. 11:668-683; DeNeve et al. (1997) Plant J. 11:15-29; and, DeBuck et al. (1998) Mol. Plant Microb Interact. 11:449-57.

The composition comprising the Agrobacterium that is injected into the embryo sac can further comprise one or more surfactants. In one example, a surfactant is added to the Agrobacterium suspension to enhance the number of embryos obtained expressing the polynucleotide of interest. A surfactant or surface-active agent refers to any compound that can reduce surface tension when dissolved in water or water solutions or that can reduce interfacial tension between a liquid (e.g., water) and a solid (e.g., bacteria). Generally, the surfactant should not be harmful to the plant, or the bacterium. Surfactants may be categorized as anionic, nonionic, zwitterionic, or cationic, depending on whether they comprise one or more charged groups. Anionic surfactants contain a negatively charged group and have a net negative charge. Nonionic surfactants contain non-charged polar groups and have no charge.

Suitable surfactants that can be used in the aqueous medium can include Triton™ brand of surfactants, the Tween™ brand of surfactants and the Silwet™ brand of surfactants. The Triton™ brand of surfactants includes specialty surfactants that are alcohols and ethoxylates, alkoxylates, sulfates, sulfonates, sulfonosuccinates or phosphate esters. One widely used non-ionic surfactant is Triton™ X-100 (t-Octylphenoxypolyethoxyethanol). Another surfactant is Silwet-L77® (polyalkyleneoxide modified heptamethyltrisiloxane). The concentration of surfactants used will vary with the type of surfactant and plant being used. Generally, surfactants are used in concentrations ranging from 0.005% to about 1% of the volume of the Agrobacterium suspension. Concentrations can also range from 0.005% to about 0.5% or from about 0.005% to about 0.05%. In some examples, a combination of compatible surfactants can be used.

Any composition of interest can be microinjected into the embryo sac, including but not limited to, DNAs (naked or coated), RNAs, mRNAs, mRNAs, oligos, siRNAs, proteins, peptides, conjugated dyes (FITC-Dextran), dyes, viral vectors, replicons, amplicons, DNA/RNA chimeras, carbohydrates, cofactors, protein complexes, any site-specific integration component, Agrobacterium, or any combination thereof. In addition, any of the various components listed above can be provided to the embryo sac using any method known for introduction, including, but not limited to, microinjection into the embryo sac, or particle bombardment, agroinfection, etc.

The methods introduce a polynucleotide into a plant via microinjection of an Agrobacterium directly into an embryo sac. The polynucleotide of interest can be either stably integrated into the genome or be transiently expressed. A transgenic plant is recovered from the microinjected embryo sac comprising the polynucleotide of interest. Stable transformation indicates that the polynucleotide of interest integrates into the genome of the plant and is capable of being inherited by the progeny thereof. Transient transformation indicates that a sequence composition is introduced into the plant and is present and/or expressed in the plant for a limited period of time.

Techniques for microinjection of various substances into a cell of interest are known. Generally, such injections occur by means of a glass microcapillary-injection pipette and employ the use of a micromanipulator (Crossway (1986) Mol. Gen. Genet. 202:179-185 and Morikawa et al. (1985) Plant Cell Physiol. 26:229-236).

Procedures for growth and suitable culture conditions for Agrobacterium are known, as well as subsequent inoculation procedures. The density of the Agrobacterium culture used for microinjection can vary from one system to the next, and therefore optimization of these parameters may be required. An effective concentration of the bacterial inoculum comprises a concentration of Agrobacterium which, when injected into the embryo sac of a plant, is sufficient to allow for the recovery of a plant expressing the polynucleotide of interest. Such concentration ranges include, for example, an Agrobacterium optical density (OD₅₅₀) of about 0.5, 0.1, 0.05, 0.01, 0.005, 0.001, or 0.0005. In some examples, the effective concentration of Agrobacterium in the inoculum is about 0.1 O.D. In other examples, the O.D. of the Agrobacterium inoculum is about 0.5 to about 0.1, about 0.1 to about 0.05, about 0.05 to about 0.01, about 0.01 to about 0.005, about 0.005 to about 0.001, or about 0.001 to about 0.0005. The concentration of the Agrobacterium in the inoculum is only limited by the highest level of bacterium that does not clog the micropipette and the lowest level of bacterium which can produce bacterial growth when dispensed in a microinjection droplet onto standard bacterial growth media. In addition, the effective concentration of Agrobacterium will be sufficient to produce the desired number of integration events and produce an acceptable integration pattern for the desired purpose.

In some examples, a dye marker can be used with the injection composition in order to confirm delivery to the nucellar slab and/or embryo sac. Typically, the dye used is a vital dye, and may be conjugated to an inert matrix. Any suitable dye, compatible with the injection parameters, Agrobacterium, and cell culture can be used. For example, a fluorescent marker such as FITC-Dextran can be co-injected with the Agrobacterium, and delivery confirmed using a fluorescence microscope.

A variety of tissue culture media, which when supplemented appropriately, support plant tissue growth and development and are suitable for plant transformation and regeneration. These tissue culture media can either be purchased as a commercial preparation or custom prepared and modified. Examples of such media include, but are not limited to, Murashige and Skoog (Murashige et al. (1962) Physiol. Plant 15:473-497), N6 (Chu et al. (1975) Scienta Sinica 18:659), Linsmaier and Skoog (Linsmaier et al. (1965) Physio. Plant. 18:100), Uchimiya and Murashige (Uchimiya et al. (1974) Plant Physiol. 15:473), Gamborg's media (Gamborg et al. (1968) Exp. Cell Res. 50:151), D medium (Duncan et al. (1985) Planta 165:322-332), McCown's Woody plant media (McCown et al. (1981) HortScience 16:453), Nitsch and Nitsch (Nitsch et al. (1969) Science 163:85-87), and Schenk and Hildebrandt (Schenk et al. (1972) Can. J. Bot. 50:199-204) or derivations of these media supplemented accordingly. Media and media supplements, such as nutrients and growth regulators for use in transformation and regeneration and other culture conditions such as light intensity during incubation, pH, and incubation temperatures, can be optimized for the particular target plant of interest.

Following microinjection, the injected embryo sacs are cultured on appropriate media. In some examples, the injected embryo sacs are initially cultured in media that contains no selection agent. Such culture conditions are referred to as a resting phase. The duration of the resting phase can vary, depending on the nature of the plant system being injected and the nature of the marker being used. In some examples, the resting phase ranges from about less than one hour to about 80 hours, for example from about 6, 8, 10, 12, 14, 16, 18, 20, 24, 26, 28, 30, 32, 34, 36, 38, 40, 42, 44, 46, 48, 50, 52, 54, 56, 58, 60, 62, 64, 66, 68, 70, 72, 73, 74, 75, 76, 77, 78, 79, or 80 hours. Typically, a resting phase of about 72 hours post-microinjection is used for embryo sacs isolated from corn.

Depending on the marker employed, injected embryos having the polynucleotide of interest can be detected in the absence of selection pressure. For example, after the embryo sac is injected with the Agrobacterium, optionally following the resting step, the embryo sacs can be analyzed for efficiency of DNA delivery by a transient assay to detect the presence of one or more polynucleotides contained on the T-DNA vector, including, but not limited to, a screenable marker gene such as β-glucuronidase (GUS), or fluorescent proteins (e.g., GFP, YFP, RFP, CFP). If GUS is used, the total number of blue spots for a selected number of embryo sacs is used as a positive correlation of DNA transfer efficiency. The efficiency of T-DNA delivery and a prediction of transformation efficiencies can be tested in transient analyses. Any number of methods are suitable for plant analyses including, but not limited to, microscopic/visual screenings, histochemical assays, biological assays, and molecular analyses.

In other examples, the injected embryo sacs are exposed to selective pressure to select for those cells that have received and are expressing the selection marker introduced by the Agrobacterium. The injected embryos are exposed to selective pressure after, or in the absence of, a resting phase. Such pressure is applied by placing the injected embryo sac directly onto selective media. The embryo sacs can be subcultured onto selective media in successive steps or stages. For example, the first selective media can contain a low amount of selective agent, and the next sub-culture can contain a higher concentration of selective agent or vice versa. The embryo sacs can also be placed directly on a fixed concentration of selective agent. Numerous modifications in selective regimes, media, and growth conditions that can be varied depending on the plant system and the selective agent. See, for example, U.S. Pat. No. 6,822,144.

In some examples, the bar gene is incorporated into a superbinary vector that is introduced into the Agrobacterium. The bar gene confers herbicide resistance to glufosinate-type herbicides, such as phosphinothricin (PPT) or bialaphos, and the like. Examples of other selective markers that could be used in the vector constructs include, but are not limited to, the pat gene, also for bialaphos and phosphinothricin resistance, the ALS gene for imidazolinone resistance, the HPH or HYG gene for hygromycin resistance, the EPSP synthase gene, ALS, GOX, or GAT gene for glyphosate resistance, the Hml gene for resistance to the Hc-toxin, and other selective agents used routinely. Additional selective agents include but are not limited to antibiotics such as geneticin (G418), kanamycin, paromycin, spectinomycin, streptomycin, or penicillin.

The cultures are subsequently transferred to a media suitable for the recovery of transformed plantlets. A number of methods are available to recover transformed plants. Additionally, a variety of media and transfer requirements can be implemented and optimized for each plant system for recovery of transgenic plants. Consequently, media and culture conditions can be modified or substituted with nutritionally equivalent components, or similar processes for selection and recovery of transgenic events.

The transformants produced are subsequently analyzed to determine the presence or absence of a particular polynucleotide. Molecular analyses can include but are not limited to Southern blots, PCR (polymerase chain reaction) analyses, immunodiagnostic approaches, and/or field evaluations. These and other well known methods can be performed to confirm the stability of the transformed plants produced by the methods. See for example, Sambrook et al. (1989) Molecular Cloning, A Laboratory Manual.

The embryo sacs that have been transformed may be grown into plants in conventional ways. See, for example, McCormick et al. (1986) Plant Cell Rep 5:81-84, Laurie et al. (1999) In Vitro Cell Dev Biol Plant 35: 320-325, and U.S. Pat. No. 6,300,543. These plants may be grown, and either pollinated with the same transformed strain or different strains, and progeny expressing the polynucleotide of interest identified. Two or more generations may be grown to ensure that expression of the polynucleotide of interest is stably maintained and inherited by the progeny, and then seeds harvested. In this manner transformed seed having the polynucleotide of interest stably incorporated into their genome are provided.

The term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Progeny, variants, and mutants of the regenerated plants comprising the introduced polynucleotide(s) are also included.

Polynucleotides can comprise deoxyribonucleotides, ribonucleotides and any combination of ribonucleotides and deoxyribonucleotides including naturally occurring molecules, modified, and/or synthetic analogues. The polynucleotides also encompass all forms of sequences including, but not limited to, single-stranded forms, double-stranded forms, hairpins, stem-and-loop structures, and the like.

The polynucleotide of interest can be provided in an expression cassettes for expression in the plant of interest. The cassette may include 5′ and 3′ regulatory sequences operably linked to a polynucleotide of interest. Operably linked denotes a functional linkage between two or more elements. For example, an operable linkage between a polynucleotide of interest and a promoter is a functional link that allows for expression of the polynucleotide of interest. Operably linked elements may be contiguous or non-contiguous. When used to refer to the joining of two protein coding regions, operably linked indicates that the coding regions are in the same reading frame. The cassette may additionally contain at least one additional gene to be cotransformed into the organism. Alternatively, additional gene(s) can be provided on multiple expression cassettes. An expression cassette may be provided with a plurality of restriction sites and/or recombination sites for insertion of the polynucleotide of interest under the transcriptional regulation of the regulatory regions. The expression cassette may additionally contain selectable marker genes.

The expression cassette can include in the 5′-3′ direction of transcription, a transcriptional and translational initiation region a polynucleotide of interest, and/or a transcriptional and translational termination region functional in plants. The regulatory regions and/or the polynucleotide of interest may be native/analogous to the host cell and/or to each other. Alternatively, the regulatory regions and/or the polynucleotide of interest may be heterologous to the host cell and/or to each other. Heterologous includes a sequence that originates from a foreign species, or, if from the same species, is substantially modified from its native form in composition and/or genomic locus by deliberate human intervention. For example, a promoter operably linked to a heterologous polynucleotide may be from a species different from the species from which the polynucleotide was derived, or, if from the same/analogous species, one or both are substantially modified from their original form and/or genomic locus, or the promoter is not the native promoter for the operably linked polynucleotide. A chimeric gene comprises a coding sequence operably linked to a transcription initiation region that is heterologous to the coding sequence. The polynucleotide of interest may be expressed using heterologous promoters, or the native promoter sequences may be used. Such constructs can change expression levels of polynucleotide of interest in the plant or plant cell. Thus, the phenotype of the plant or plant cell can be altered.

The termination region may be native with the transcriptional initiation region, may be native with the operably linked polynucleotide of interest, may be native with the plant host, or may be derived from another source than the promoter, the polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be modified for increased expression in the transformed plant. For example, the polynucleotides can be synthesized using plant-preferred codons for improved expression. See, for example, Campbell and Gowri (1990) Plant Physiol. 92:1-11 for a discussion of host-preferred codon usage. Methods for synthesizing plant-preferred genes include, for example, U.S. Pat. Nos. 5,380,831, and 5,436,391, and Murray et al. (1989) Nucleic Acids Res. 17:477-498.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. The sequence may also be modified to avoid predicted hairpin secondary mRNA structures.

The expression cassettes may additionally contain 5′ leader sequences. Such leader sequences can act to enhance translation. Translation leaders include picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Allison et al. (1986) Virology 154:9-20; and Kong et al. (1988) Arch. Virol. 143:1791-1799), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated to provide the DNA sequences in the proper orientation and, as appropriate, in the proper reading frame. Adapters and/or linkers may be employed to join the DNA fragments, and/or other manipulations to provide convenient restriction sites, removal of superfluous DNA, removal of restriction sites, or the like may be used. For this purpose, in vitro mutagenesis, primer repair, restriction, annealing, resubstitutions, transitions and transversions, may be involved.

A number of promoters can be used, for example, the polynucleotide(s) of interest can be combined with constitutive, tissue-preferred, or other promoters for expression in plants. For a review of promoters useful in plants see, for example, Potenza et al. (2004) In Vitro Cell Dev Biol 40:1-22.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); PEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Pathogen-inducible promoters induced following infection by a pathogen include, but are not limited to, those regulating expression of PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; Van Loon (1985) Plant Mol. Virol. 4:111-116; WO 99/43819; Marineau et al. (1987) Plant Mol. Biol. 9:335-342; Matton et al. (1989) Mol Plant Microbe Interact 2:325-331; Somsisch et al. (1986) Proc. Natl. Acad. Sci. USA 83:2427-2430; Somsisch et al. (1988) Mol. Gen. Genet. 2:93-98; Yang (1996) Proc. Natl. Acad. Sci. USA 93:14972-14977; Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein.

Wound-inducible promoters include potato proteinase inhibitor (pin 11) gene (Ryan (1990) Ann. Rev. Phytopath. 28:425-449; Duan et al. (1996) Nat Biotech 14:494-498); wun1 and wun2 (U.S. Pat. No. 5,428,148); win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like.

Chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzenesulfonamide herbicide safeners; the maize GST promoter, activated by hydrophobic electrophilic compounds used as pre-emergent herbicides; and the tobacco PR-1a promoter, activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14:247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Tissue-preferred promoters can be utilized to target enhanced expression of a polynucleotide of interest within a particular plant tissue. Tissue-preferred promoters include Kawamata et al. (1997) Plant Cell Physiol. 38:792-803; Hansen et al. (1997) Mol. Gen Genet. 254:337-343; Russell et al. (1997) Transgenic Res. 6:157-168; Rinehart et al. (1996) Plant Physiol. 112:1331-1341; Van Camp et al. (1996) Plant Physiol. 112:525-535; Canevascini et al. (1996) Plant Physiol. 112:513-524; Lam (1994) Results Probl. Cell Differ. 20:181-196; and Guevara-Garcia et al. (1993) Plant J. 4:495-505.

Leaf-preferred promoters are known and include, for example, Yamamoto et al. (1997) Plant J. 12:255-265; Kwon et al. (1994) Plant Physiol. 105:357-67; Yamamoto et al. (1994) Plant Cell Physiol. 35:773-778; Gotor et al. (1993) Plant J. 3:509-18; Orozco et al. (1993) Plant Mol. Biol. 23:1129-1138; and Matsuoka et al. (1993) Proc. Natl. Acad. Sci. USA 90:9586-9590.

Root-preferred promoters are known and include, for example, Hire et al. (1992) Plant Mol. Biol. 20:207-218 (soybean root-specific glutamine synthetase gene); Miao et al. (1991) Plant Cell 3:11-22 (cytosolic glutamine synthetase (GS) expressed in roots and root nodules of soybean); Keller and Baumgartner (1991) Plant Cell 3:1051-1061 (root-specific control element in the GRP 1.8 gene of French bean); Sanger et al. (1990) Plant Mol. Biol. 14:433-443 (root-specific promoter of the mannopine synthase (MAS) gene of A. tumefaciens); Bogusz et al. (1990) Plant Cell 2:633-641 (root-specific promoters isolated from Parasponia andersonii and Trema tomentosa); Leach and Aoyagi (1991) Plant Sci. 79:69-76 (roIC and roID root-inducing genes of A. rhizogenes); Teeri et al. (1989) EMBO J. 8:343-350 (Agrobacterium wound-induced TR1′ and TR2′ genes); VfENOD-GRP3 gene promoter (Kuster et al. (1995) Plant Mol. Biol. 29:759-772); and rol B promoter (Capana et al. (1994) Plant Mol. Biol. 25:681-691. See also U.S. Pat. Nos. 5,837,876; 5,750,386; 5,633,363; 5,459,252; 5,401,836; 5,110,732; and 5,023,179.

Seed-preferred promoters include both seed-specific promoters active during seed development, as well as seed-germinating promoters active during seed germination. See Thompson et al. (1989) BioEssays 10:108. Seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529). For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, gamma-zein, waxy, shrunken 1, shrunken 2, globulin 1, etc. See also WO 00/12733, where seed-preferred promoters from end1 and end2 genes are disclosed.

The expression cassette can also comprise a screenable marker gene. Screenable marker genes are utilized for the identification and/or selection of transformed cells or tissues, and include markers that confer resistance or sensitivity to a compound, visual/screenable markers, and the like. Any marker can be used including genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase, β-glucuronidase (GUS), and fluorescent proteins such as green fluorescent protein (GFP) (Su et al. (2004) Biotechnol Bioeng 85:610-9 and Fetter et al. (2004) Plant Cell 16:215-28), cyan fluorescent protein (CFP) (Bolte et al. (2004) J. Cell Sci. 117:943-54 and Kato et al. (2002) Plant Physiol 129:913-42), red fluorescent protein, and yellow fluorescent protein (e.g., ZsYellow from InVitrogen, or PhiYFP™ from Evrogen, see, Bolte et al. (2004) J. Cell Sci. 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724.

The methods can be used for modulating the concentration and/or activity of the polynucleotide of interest in a plant, and in some examples, the polypeptide it encodes. In general, concentration and/or activity is increased or decreased by at least about 1%, 5%, 10%, 20%, 30%, 40%, 50%, 60%, 70%, 80%, 90%, 95%, or about 100% relative to a native control plant, plant part, or cell which did not have the polynucleotide of interest introduced. In some examples, concentration and/or activity is increased less than about 1-fold, 1-fold (1×), 2×, 3×, 5×, 7×, 10×, or greater than 10×. Modulation may occur during and/or subsequent to growth of the plant to the desired stage of development. In specific examples, the polynucleotides of interest are modulated in monocots, for example, maize.

The expression level of the polynucleotide of interest may be measured directly, for example, by assaying for the level of the polynucleotide or the protein encoded thereby in the plant, or indirectly, for example, by measuring the activity of the polypeptide in the plant.

Methods for inhibiting or eliminating the expression of a gene in a plant are well known. Reduction of the activity of specific genes may be desirable for several aspects of genetic engineering in plants. Many techniques for gene silencing are known, including, but not limited to, antisense technology (see, e.g., Sheehy et al. (1988) Proc. Natl. Acad. Sci. USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8:340-344; Flavell (1994) Proc. Natl. Acad. Sci. USA 91:3490-3496; Finnegan et al. (1994) Bio/Technology 12:883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) Proc. Natl. Acad. Sci. USA 95:15502-15507), virus-induced gene silencing (Burton et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; patent publications US20030175965, US20030180945, WO 99/53050, WO 02/00904, and WO 98/53083; Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. (2003) BMC Biotechnology 3:7; Panstruga et al. (2003) Mol. Biol. Rep. 30:135-140; Wesley et al. (2001) Plant J. 27:581-590; Wang and Waterhouse (2001) Curr. Opin. Plant Biol. 5:146-150; ribozymes (Steinecke et al. (1992) EMBO J. 11:1525; and Perriman et al. (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345; and WO 00/42219); transposon tagging (Maes et al. (1999) Trends Plant Sci. 4:90-96; Dharmapuri and Sonti (1999) FEMS Microbiol. Lett. 179:53-59; Meissner et al. (2000) Plant J. 22:265-274; Phogat et al. (2000) J. Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928; Bensen et al. (1995) Plant Cell 7:75-84; Mena et al. (1996) Science 274:1537-1540; and U.S. Pat. No. 5,962,764); and other methods or combinations of methods known to those of skill in the art. Any sequence that can decrease the expression of a target polynucleotide can be contained in the T-DNA of the Agrobacterium which is microinjected into the embryo sac such as sequences that produce mRNA, sRNA, antisense RNA, etc., or combinations thereof.

The methods can be employed to provide any polynucleotide of interest, or combinations of polynucleotides, to the plant. Various changes of interest include modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, for example, enzymes or cofactors in the plant. These changes may or may not result in a change in phenotype of the transformed plant, a plant part or tissue, and/or transformed seed of a part thereof.

General categories of polynucleotides of interest include, for example, those genes involved in information, such as zinc fingers, those involved in communication, such as kinases, and those involved in housekeeping, such as heat shock proteins. More specific categories of transgenes include for example genes encoding traits for agronomics, insect resistance, disease resistance, herbicide resistance, sterility, grain characteristics, and commercial products. Polynucleotides of interest include, generally, those involved in oil, starch, protein, carbohydrate, or nutrient metabolism, as well as, those affecting kernel size, sucrose loading, and the like.

Traits such as oil, starch, and protein content can be genetically altered. Modifications include increasing content of oleic acid, saturated and unsaturated oils, increasing levels of lysine and sulfur, providing essential amino acids, and modification of starch. Hordothionin protein modifications are described in U.S. Pat. Nos. 5,703,049, 5,885,801, 5,885,802, and 5,990,389. Another example is a lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106.

Derivatives of the coding sequences can be made to increase the level of preselected amino acids in the encoded polypeptide. For example, the polynucleotides encoding the barley high lysine polypeptide (BHL) are derived from barley chymotrypsin inhibitor (WO 98/20133). Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502); corn (Pedersen et al. (1986) J. Biol. Chem. 261:6279; Kirihara et al. (1988) Gene 71:359); and rice (Musumura et al. (1989) Plant Mol. Biol. 12:123).

Insect resistance polynucleotides may encode resistance to pests such as rootworm, cutworm, European Corn Borer, and the like. Such polynucleotides include, for example, Bacillus thuringiensis toxic protein genes (U.S. Pat. Nos. 5,366,892; 5,747,450; 5,736,514; 5,723,756; and 5,593,881; Geiser et al. (1986) Gene 48:109); and the like.

Polynucleotides encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides, such as chlorosulfuron (e.g., the S4 and/or Hra mutations in ALS), genes coding for resistance to herbicides that act to inhibit action of glutamine synthase, such as phosphinothricin or basta (e.g., the bar gene); glyphosate (e.g., the EPSPS gene and the GAT gene; see, for example, patent publications US20040082770 and WO 03/092360); or other such genes known. Antibiotic resistance can also be provided, for example the nptll gene encodes resistance to the antibiotics kanamycin and geneticin.

Sterility polynucleotides can also be encoded in an expression cassette and provide an alternative to physical detasseling. Examples of polynucleotides used in such ways include male tissue-preferred genes and genes with male sterility phenotypes such as QM, described in U.S. Pat. No. 5,583,210. Other polynucleotides include kinases and those encoding compounds toxic to either male or female gametophytic development.

Commercial traits can also be encoded on a gene or genes that could increase for example, starch for ethanol production, or provide expression of proteins. Another commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like.

### EXPERIMENTAL

### Example 1

### Bacterial Manipulations

Experiments were done to demonstrate that Agrobacterium can be dispensed through the tip of a microinjection micropipette and remain viable, as evidenced by subsequent growth on plates of 810 standard solid bacterial medium.

An upper concentration limit equivalent to an OD₅₅₀ reading of 0.1 was established, above which the bacteria tended to clog the micropipette tips. A lower concentration limit equivalent to an O.D. reading of 0.0005 was established, below which no bacterial growth was observed from microinjection droplets dispensed onto 810 standard solid bacterial medium. In order to maximize the number of Agrobacterium delivered in each injection, concentrations equivalent to O.D. readings of 0.1 and 0.05 were selected for subsequent injections.

Optionally, a dye marker is added to the composition comprising the bacterial suspension with any other components to be injected. For example, FITC-Dextran (Sigma, FD-4), prepared as a 5% w/v solution in distilled water, was mixed 1:1 v/v with the bacterial composition for injection. The dextran minimizes diffusion of the dye, allowing visual confirmation of delivery of the composition in nucellar slabs using a fluorescence microscope. No significant plant recovery frequency or developmental differences were observed for embryo sac development from injected material when FITC-Dextran was included.

### Example 2

### Iniection of Maize Embrvo Sacs with Agrobacterium

Nucellar slabs were produced from kernels isolated from N46 corn inbred plants at 1 day after pollination, according to the protocols outlined in Laurie et al. (1999) In Vitro Cell Dev Biol Plant 35: 320-325. The pericarp was removed from each slab prior to injection.

Agrobacterium suspensions were provided and injections carried out with an Eppendorf FemtoJet microinjector, using pre-pulled glass capillary pipettes (Femtotipsil). For each pipette, the injection pressure was adjusted to allow the smallest possible droplet to be extruded consistently from the tip of the pipette, whilst injection time and compensation pressure were kept at 0.1 s and 50 hpa, respectively.

Both injected and non-injected slabs were cultured on 586P (co-cultivation medium; 586M+100 mM acetosyringone) and maintained in the dark at 26° C. (standard embryo sac culture conditions). Developing embryo-like structures were transferred to hormone-free 272V medium in the light, to allow maturation of shoots and roots. Plantlets were transferred to tubes of 601 G, and subsequently transferred to pots and grown to maturity in the greenhouse.

A total of 139 embryo sacs were injected and 82 kept as non-injected controls. Suspensions of Agrobacterium containing the plasmids PHP15325 (ubi::GFP) or PHP10525 (ubi::GUS) were tested at various concentrations. Injection pressures ranged between about 1000 to about 1800 hPa. In each experiment, droplets of the Agrobacterium suspensions were also expelled from the injection needle onto the surface of 810 solid medium, before and after injection of the slabs, to check for bacterial growth.

Material was monitored for any effects of injection on subsequent development and plant regeneration, and for evidence of GFP or GUS expression. A total of ten cultures (from 3 experiments) were stained for GUS expression at 4-5 weeks after injection.

Results: No significant plant recovery frequency or developmental differences were observed for embryo sac development from injected material relative to the non-injected controls. Bacterial overgrowth was observed on only 9 of the 139 embryo sacs/slabs in 2/8 experiments, at 2.5-6 weeks after injection. A summary of treatments, culture response, and plant recovery is presented in Tables 1 and 2.

TABLE 1

| | Culture | |
|---|---|---|
| Treatment | Response | Plant Recovery |
| Non-injected controls | 52/82 = 63% | 17/82 = 21% |
| Agro injections @ OD 0.1 | 38/60 = 63% | 14/60 = 23% |
| Agro injections @ OD 0.05 | 35/51 = 69% | 11/51 = 22% |

TABLE 2

| | Agrobacterium Density (O.D.) | | | | | |
|---|---|---|---|---|---|---|
| Plasmid | 0.005 | 0.001 | 0.05 | 0.1 | Total | Shoots |
| PHP15325 | 20 | 8 | 9 | 9 | 46 | 22 |
| PHP10525 | — | — | 42 | 51 | 93 | 32 |
| Control | N/A | N/A | N/A | N/A | 82 | 24 |

### Example 3

### Microiniection of Maize Embryo Sacs with Agrobacterium

We observed that the membrane surrounding the embryo sac, enclosing it within the nucellus, is relatively impermeable to a number of commonly used dyes or stains (e.g. DAPI), up to about 5 or 6 days after pollination. Additional observations with dyes and fluorescent proteins indicate that movement between the individual cells within the embryo sac occurs more freely. This suggests that this outer membrane may provide a tougher physical barrier than most cell membranes, which could compromise the ability of Agrobacterium T-DNA to enter the embryo sac by natural processes. Delivery of Agrobacterium into the embryo sac by injection should overcome problems in penetrating the membrane.

Materials: Fertilized N46 corn inbred embryos are generated by crossing a transgenic N46 line with non-transgenic N46, either line can be used as the female. Transgenic lines comprising excision activated GUS marker constructs were employed:

### PHP10006 Ubi::FRT1::GFP::35S::MOPAT::FRT1::GUS::FRT5; or,

### PHP5869 CAMV35S ENH−CAMV35S PRO−ADH1 INTRON−(ATG)::FRT1::BAR−PINII+SPACE+(TGA)::FRT1-GUS-PINII.

The ears were harvested at 1 DAP. Kernels were sectioned with a Vibratome at 250 μm. Pericarps were removed from the slabs.

If the embryos are isolated from ears pollinated by a transgenic line comprising a fluorescent marker expressed in pollen, the putative pollinated embryos can be identified by screening for the pollen marker. For example, when pollen donors comprising PHP18098 (Ubi::ubi intron::AmCyan::pinil), or PHP18096 (ubi::ZsYellow::pinil) were used, the pollen or pollen tube was visible in nucellar slabs.

Vectors: The vectors employed in the Agrobacterium were:

### PHP22462 actin-GUS; or,

### PHP17797 Ubi-FRT1::FLPm::35S::Bar::FRT5.

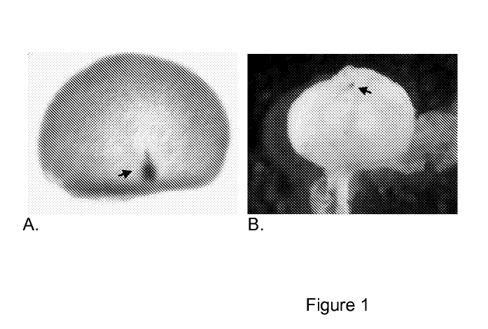

Methods: Agrobacterium containing PHP22462 or PHP17797 were grown on 810 media for 1 day at 28° C. Agrobacterium suspension diluted with 586Q to OD₅₅₀ 0.1 were prepared and injected into the embryo sacs with the microinjector. The following parameters were used with a micropipette type Femtotips II: an injection pressure of 1300 hPa, injection time of 0.1s, a compensation pressure 50 hPa. After injection, the slabs were cultured on 586P media for 1 to 4 days at 26° C., then transferred to 586N. The results are presented in Table 3 and FIG. 1 (B).

TABLE 3

| | | | # Slabs | # Slabs |
|---|---|---|---|---|
| | Materials | Vectors | Injected | GUS+ |
| Exp #1 | N46-78 × N46 | PHP22462 | 22 | 1 |
| Exp #2 | N46-78 × N46 | PHP22462 | 22 | 2 |
| Exp #3 | N46-78 × N46 | PHP22462 | 20 | 6 |
| Exp #4 | N46 × PHP10006 | PHP17797 | 32 | 1 |

FIG. 1 shows:
A. (Left): An immature kernel section (slab) from a 1-day post-pollinated immature kernel containing an embryo sac that was microinjected with Agrobacterium. The T-DNA within the injected Agrobacterium comprises Actin promoter::GUS (PHP22462). Following injection of the Agrobacterium, expression of GUS gene in the embryo sac (dark heart-shaped at the bottom of the section, arrow) indicates that the functional Agrobacterium has been delivered and the GUS gene on the T-DNA was expressed. Bar=0.5 mm
B. (Right): An embryo/endosperm structure derived from a microinjected 1-day post-pollinated slab after 21 days in vitro culture. The nucellar slab isolated from a transgenic plant stably transformed with PHP10006 (Ubi::FRT1::GFP::35S::MOPAT::FRT1::GUS::FRT5) was injected with Agrobacterium containing PHP17797 (RB::Ubi-FRT1::FLPm::35S::Bar::FRT5::LB). The FLP enzyme expressed from PHP17797 mediated excision of the DNA between the FRT1 sites in PHP10006 in the injected slab, thereby creating an operable linkage of the promoterless GUS gene to the Ubi promoter, resulting in GUS expression. The blue spot (arrow) shows the GUS expression in the embryo area, demonstrating T-DNA transfer and gene expression from the injected Agrobacterium. Bar=1 mm.

### Example 4

### Cell culture and regeneration media

Medium 586M comprises 4.3 g/L MS salts (Gibco 11117-074), 5 ml/L MS Vitamins (0.100 g/L nicotinic acid, 0.02 g/L thiamine HCl, 0.10 g/L pyridoxine HCl, and 0.40 g/L glycine brought to volume with D-1 H₂O), 0.5 mg/L thiamine HCl, 0.4 g/L asparagine, 0.1 mg/L BAP (6-benzylaminopurine), and 150 g/L sucrose (brought to volume with D-1 H₂O following adjustment to pH 5.8 with KOH); and 3 g/L Gelrite (added after bringing to volume with D-1 H₂O).

Culture medium 586P comprises medium 586M+100 mM acetosyringone.

Culture medium 586N comprises medium 586M+100 mg/L carbenicillin (added after sterilizing the medium and cooling to room temperature).

Medium 810 comprises 950 ml D-1 H₂O, 5 g/L yeast extract (DIFCO), 10 g/L peptone (DIFCO), and 5 g/L NaCl (brought to volume with D-1 H₂O after adjustment to pH 6.8); and 15 g/L Bacto-Agar (added after bringing to volume with D-1 H₂O); and 50 mg/L spectinomycin (added after sterilizing the medium and cooling to room temperature).

Medium 601 G comprises 2.15 g/L MS salts (GIBCO 11117), 5 ml/L MS vitamins stock solution (0.100 g/L nicotinic acid, 0.02 g/L thiamine HCl, 0.10 g/L pyridoxine HCl, and 0.40 g/L glycine brought to volume with polished D-1 H₂O), 0.1 g/L myo-inositol, and 40.0 g/L sucrose (brought to volume with polished D-1 H₂O after adjusting pH to 5.6), 0.7 ml/L 1 mg/ml stock IBA, 0.3 ml/L 1 mg/ml stock NAA, (brought to volume with D-1 H₂O after adjusting pH to 5.8); and 1.5 g/L Gelrite, sterilized and cooled to 60° C.

Medium 272V comprises 4.3 g/L MS salts (GIBCO 11117-074), 5.0 ml/L MS vitamins stock solution (0.100 g/L nicotinic acid, 0.02 g/L thiamine HCl, 0.10 g/L pyridoxine HCl, and 0.40 g/L glycine brought to volume with D-1 H₂O), 0.1 g/L myo-inositol, and 40.0 g/L sucrose (brought to volume with D-1 H₂O after adjusting pH to 5.6); and 6 g/L bacto-agar (added after bringing to volume with D-I H₂O), sterilized and cooled to 60° C.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

What is claimed is:

1. A method for expressing a polynucleotide of interest in a plant comprising
a) providing an embryo sac from the plant;
b) injecting into the embryo sac a composition comprising an effective concentration of an Agrobacterium comprising a T-DNA comprising the polynucleotide of interest operably linked to a promoter active in the plant, wherein said Agrobacterium is capable of T-DNA transfer into a plant cell.

2. The method of claim 1, further comprising recovering from the embryo sac a transgenic plant having the polynucleotide of interest stably integrated into its genome.

3. The method of claim 1, wherein the embryo sac comprises a fertilized embryo sac.

4. The method of claim 3, wherein the fertilized embryo sac comprises an embryo or a zygote.

5. The method of claim 1, wherein the plant is a monocot or a dicot.

6. The method of claim 5, wherein said plant is selected from the group consisting of maize, barley, millet, wheat, rice, soybean, canola, alfalfa, sunflower, safflower, tobacco, Arabidopsis, and cotton.

7. The method of claim 1, wherein the Agrobacterium comprises Agrobacterium tumefaciens.

8. A method of introducing an Agrobacterium into a plant comprising
a) providing an embryo sac from the plant;
b) injecting into the embryo sac a composition comprising an effective concentration of an Agrobacterium, wherein the Agrobacterium is capable of T-DNA transfer into a plant cell.

9. The method of claim 8, wherein the Agrobacterium comprises a T-DNA comprising a polynucleotide of interest operably linked to a promoter active in the plant.

10. The method of claim 9, further comprising recovering from the embryo sac a transgenic plant having the polynucleotide of interest stably integrated into its genome.

11. The method of claim 8, wherein the embryo sac comprises a fertilized embryo sac.

12. The method of claim 11, wherein the fertilized embryo sac comprises an embryo or a zygote.

13. The method of claim 8, wherein the plant is a monocot or a dicot.

14. The method of claim 13 wherein the plant is selected from the group consisting of maize, barley, millet, wheat, rice, soybean, canola, alfalfa, sunflower, safflower, tobacco, Arabidopsis, and cotton.

15. The method of claim 8 wherein the Agrobacterium comprises Agrobacterium tumefaciens.

16. A method to identify a fertilized plant embryo sac comprising
a) providing pollen from a first plant comprising a polynucleotide encoding a visual marker operably linked to a promoter, wherein promoter is active in the pollen or in an embryo sac;
b) providing a population of unfertilized seed, each seed comprising an embryo sac;
c) contacting the seed with the pollen; and
d) identifying the fertilized embryo sac expressing said visual marker.

17. The method of claim 16, wherein the visual marker is expressed in a central cell of the embryo sac, a zygote of the embryo sac, the pollen, or a pollen tube.

18. The method of claim 16, wherein the visual marker is a fluorescent protein.

19. The method of claim 16, wherein the visual marker is encoded by a polynucleotide having maize preferred codons.

20. The method of claim 16, wherein the fertilized plant embryo is from a monocot.
